(19) United States
(12) Patent Application Publication
(10) Pub. No.: US 2001/0006788 A1
(43) Pub. Date: Jul. 5, 2001

(54) IDENTIFYING THE ANTIGENIC TARGET OF AUTOIMMUNE SENSORINEURAL HEARING LOSS (AISNHL) AND DEVELOPEMENT OF SPECIFIC TESTS FOR DIAGNOSIS AND MANAGEMENT OF AISNHL

(76) Inventors: THOMAS E. CAREY, DEXTER, MI (US); THANKUM S. NAIR, ANN ARBOR, MI (US); JENNIFER P. GRAY, YPSILAMT, MI (US)

Correspondence Address: PETER G CARROLL, MEDLEN AND CARROLL, 220 MONTGOMERY STREET, SUITE 2200, SAN FRANCISCO, CA, 94104

(21) Appl. No.: 09/222,179

(22) Filed: Dec. 29, 1998

(51) Int. Cl. G01N 33/53; A61K 39/00; C07K 14/00; C07K 16/00

(52) U.S. Cl. 435/7.2; 424/185.1; 530/388.2; 530/395

(57) ABSTRACT

The substantial purification and characterization of the glycoprotein Inner Ear Supporting Cell Antigen (IESCA) is described, along with methods employing IESCA to detect autoimmune sensorineural hearing loss (AISNHL), agonists and antagonists of IESCA binding and anti-IESCA binding, as well as homologs of IESCA.

This invention was made in part with government support under grant DC 002272 from the National Institutes of Health. The government has certain rights in the invention.

### FIELD OF THE INVENTION

This invention generally relates to a novel antigen, Inner Ear Supporting Cell Antigen (IESCA), reactive with an autoantibody associated with autoimmune sensorineural hearing loss (AISNHL) and methods for the detection of AISNHL in a patient. Additionally, a kit containing reagents to assay for an antibody associated with AISNHL in a patient is disclosed and claimed by this invention. Furthermore, drugs screens for compounds that are agonistic or antagonistic to IESCA binding, as well as agonistic or antagonistic to anti-IESCA antibody binding, are claimed by this invention. Also, screens for IESCA homologs are claimed by this invention. The invention represents a major improvement over existing tests for AISNHL which identify antibodies to a universally distributed substance that is not unique to the inner ear and has never been linked to hearing loss.

### BACKGROUND

Sensorineural Hearing Loss. Sensorineural hearing loss (SNHL) is a common disorder affecting millions of Americans. SNHL is the result of damage to either the sensory system within the inner ear or the nerves that carry information from the sensory system to the brain. The inner ear is a tiny organ comprised of specialized sensory cells. These specialized cells are called hair cells because they have stereocilia or long stiff projections from their upper surface. The hair cells are arranged in the inner ear or cochlea within the organ of Corti, in order from the high frequency region in the base of the cochlea to the low frequency region in the apex. Sounds are transmitted from the eardrum to the inner ear by small bones called ossicles. These bones vibrate against a membrane in the cochlea transmitting the energy to the fluid inside. The fluid moves the organ of Corti stimulating hair cells at the appropriate frequency. The stimulated hair cells then stimulate nerves that send the information to the brain for processing. If hair cells are damaged or lost then hearing is affected for the frequencies encoded by those sensory cells.

Autoimmune Hearing Loss In Humans. Unexplained or idiopathic SNHL is troubling to physicians and patients alike because the etiology is unknown and there are few effective treatments. Autoimmunity is suspected to be a cause of some cases of sudden onset, rapidly progressive or fluctuating hearing loss, particularly when bilateral involvement occurs. Autoimmune sensorineural hearing loss (AISNHL) in humans has been suspected in systemic autoimmune diseases, and there are indications that inner ear organ-specific autoimmunity is involved in rapidly progressive hearing loss (Harris J P, “Immunologic Mechanisms In Disorders Of The Inner Ear,” In: 2nd edition Otolaryngology Head and Neck Surgery, Vol IV: Ear and Cranial Base, Cummings C W, Krause C J, Schuller D E, Fredrickson J M, Harker L A (eds), Mosby Yearbook, St. Louis, Mo., pp. 2926-2942, 1993; McCabe B F, “Autoimmune Sensorineural Hearing Loss,” Ann. Otol., 88:585-859, 1979; McCabe B F, “Autoimmune Inner Ear Disease: Results Of Therapy,” In: Bearing Of Basic Research On Clinical Otolaryngology, Adv. Otorhinolaryngol, Pfaltz C R, Arnold W, Kleinsasser O (eds), Karger Publishing, Basel, Switzerland, Vol. 46, pp. 78-81, 1991; Hughes et al. “Clinical Diagnosis Of Immune Inner-Ear Disease,” Laryngoscope, 98:251-253, 1988; Harris and Ryan “Immunobiology Of The Inner Ear,” Am. J Otolaryngol, 5:418-425, 1984; Cruz et al., “Autoimmune Sensorineural Hearing Loss: A Preliminary Experimental Study,” Am. J. Otol, 11:342-346, 1990; Harris, “Immunology Of The Inner Ear: Response Of The Inner Ear To Antigen Challenge,” Otolaryngol Head Neck Surg., 91:17, 1983; Arnold et al., “Evidence Of Serum Antibodies Against Inner Ear Tissues In The Blood Of Patients With Certain Sensorineural Hearing Disorders,” Acta Otolaryngol, 99:437, 1985; Harris and Sharp, “Inner Ear Autoantibodies In Patients With Rapidly Progressive Sensorineural Hearing Loss,” Laryngoscope, 100:516-524, 1990; Moscicki et al., “Serum Antibody To Inner Ear Proteins In Patients With Progressive Hearing Loss: Correlation With Disease Activity And Response To Corticosteroid Treatment,” JAMA, 272:611-616, 1994; Sismanis et al., “Methotrexate Management Of Immune-Mediated Cochleovestibular Disorders,” Otolaryngol Head Neck Surg., 116:146-152, 1997).

Given the high frequency of idiopathic SNHL, the ability to accurately diagnose autoimmune SNHL (AISNHL) would be of value since this is one of the few potentially treatable causes of SNHL. However, since treatment of autoimmune disease involves toxic drugs such as corticosteroids, cyclophosphamide, methotrexate and cyclosporin A, all of which have significant side effects, most physicians are reluctant to use these agents without a clear indication (Sismanis et al., “Methotrexate Management Of Immune-Mediated Cochleovestibular Disorders,” Otolaryngol Head Neck Surg., 116:146-152, 1997). Treatment of suspected autoimmune hearing loss is complicated because without treatment there is a high frequency of spontaneous remission, but in cases that don't regress, the hearing loss may become worse and permanent. Immune-mediated hearing loss also may include 30-50% of Meniere's disease (episodic vertigo and fluctuating progressive hearing loss) patients (Rauch et al., “Serum Antibodies Against Heat Shock Proteins In Meniere's Disease,” Am. J. Otology, 16:648-652, 1995; Shin et al., “Comparison Of Anti-Heat Shock Protein 70 (Anti-hsp70) And Anti-68 kDa Inner Ear Protein In The Sera Of Patients With Meniere's Disease,” Laryngoscope, 107:222-227, 1997).

Current State Of The Art. Early approaches to detecting AISNHL employed cellular assays of immune reactivity such as lymphocyte transformation and lymphocyte migration inhibition assays using crude inner ear antigens (McCabe B F, “Autoimmune Inner Ear Disease: Results Of Therapy,” In: Bearing Of Basic Research On Clinical Otolaryngology, Adv. Otorhinolaryngol, Pfaltz C R, Arnold W, Kleinsasser O (eds), Karger Publishing, Basel, Switzerland, Vol. 46, pp. 78-81, 1991; Hughes et al., “Practical Versus Theoretical Management Of Autoimmune Inner Ear Disease,” Laryngoscope, 94:758-767, 1984). Unfortunately, these assays did not correlate well with response to therapy (Hughes et al., “Practical Versus Theoretical Management Of Autoimmune Inner Ear Disease,” Laryngoscope, 94:758-767, 1984; Kanzaki J and O-Uchi T, “Circulating Immune Complexes In Steroid-Responsive Sensorineural Hearing Loss And The Long-Term Observation,” Acta. Otolaryngol. (suppl.), 393:77-84, 1983; Hughes et al., “Clinical Diagnosis Of Immune Inner-Ear Disease,” Laryngoscope, 98:251-253, 1988; Veldman et al., “Autoimmunity And Inner Ear Disorders: An Immune-Complex Mediated Sensorineural Hearing Loss,” Laryngoscope, 94:501-507, 1984). The poor predictability has been explained by a lack of sensitivity and specificity of these assays for identifying organ-specific autoimmune reactivity (Mattox and Lyles, “Idiopathic Sudden Sensorineural Hearing Loss,” Am. J. Otol., 10:242-247, 1989; Mattox and Simmons, “Natural History Of Sudden Sensorineural Hearing Loss,” Ann. Otol. Rhinol. Laryngol., 86:463-480, 1977). Currently, there is general consensus among researchers in the field that the target antigen of the hearing loss antibody is a 68 kD protein found in the inner ear. It has been postulated that the protein is HSP-70 (Billings et al., “Evidence Linking The 68 Kilodalton Antigen Identified In Progressive Sensorial Hearing Loss Patient Sera With Heat Shock Protein 70,” Ann. Otol. Rhinol. Laryngol., 104:181-188, 1995). Dr. Harris patented the use of a Western blot for HSP-70 as a diagnostic tool for AISNHL (U.S. Pat. No. 5,422,282) and worked with OtoImmune (IMMCO Diagnostics) to bring a product to market based on his research. However, the correlation between the Western blot result and a patient responding to steroid treatment is low, both in our lab (53%), and in the literature (Moscicki et al., “Serum Antibody To Inner Ear Proteins In Patients With Progressive Hearing Loss: Correlation With Disease Activity And Response To Corticosteroid Treatment,” JAMA, 272:611-616, 1994). What is needed are more accurate tests to detect immune-mediated hearing loss with the goal of selecting patients who have a greater likelihood of responding to steroid treatment.

### SUMMARY OF THE INVENTION

The present invention generally comprises a novel, substantially purified antigen from the organ of Corti reactive with autoantibodies and monoclonal antibodies (MAb) associated with or relating to autoimmune sensorineural hearing loss. The antigen is named inner-ear supporting cells antigen (IESCA). IESCA has a molecular weight in the range of about 68,000-70,000 daltons as determined by SDS-PAGE analysis under reducing conditions and 65,000-68,000 daltons under non-reducing conditions and has reactivity to auto-antibodies associated with AISNHL. IESCA has been isolated with anti-IESCA MAb KHRI-3 both by immunoprecipitation and antibody affinity chromatography. The invention also comprises purified epitopes of IESCA having reactivity with the autoantibodies associated with AISNHL. IESCA is different from another 68 kD antigen previously identified by Harris and coworkers (Harris and Sharp, “Inner Ear Antibodies In Patients With Rapidly Progressive Sensorineural Hearing Loss,” Laryngoscope, 100:516-524, 1990) and associated with AISNHL. That antigen was later identified to be heat shock protein-70 (HSP-70) (Billings et al., “Evidence Linking The 68 Kilodalton Antigen Identified In Progressive Sensorineural Hearing Loss Patient Sera With Heat Shock Protein 70,” Laryngoscope, 105:1347-1352, 1995; Shin et al., “Comparison Of Anti-Heat Shock Protein (Anti-hsp70) And Anti-68-kDa Inner Ear Protein In The Sera Of Patients With Meniere's Disease,” Laryngoscope, 107:222-227, 1997). Immunoprecipitation experiments have demonstrated that the antibodies to either IESCA or HSP-70 will not cross react to antigens immunoprecipitated by the other antibody.

Additionally, the invention comprises the amino acid, DNA and RNA sequences, and portions thereof, necessary for the production of IESCA and portions of IESCA, as well as antibodies generated from the protein sequences (and portions thereof), and expression constructs and transgenic animals generated from the nucleotide sequences (and portions thereof). Exemplary N-terminal amino acid sequences (SEQ ID NO:1 and SEQ ID NO:2) are presented in FIG. 1 and FIG. 2, respectively, where single letter nomenclature is used; where X means any amino acid; where question mark (?) means amino acid not yet determined; where a single letter amino acid designation followed by a question mark (?) means probably, though not necessarily, the designated amino acid; and where single letter amino acid designations separated by forward slashes (/) means either of the amino acids separated by the slash can occupy that position.

The invention also contemplates novel compositions. A first composition comprises isolated guinea pig (or other suitable animal) organ of Corti (or other suitable tissue that contains IESCA) and human sera from individuals suspected of having AISNHL. A second composition comprises extracts from guinea pig (or other suitable animal) organ of Corti (or other suitable tissue that contains IESCA), KHRI-3 (a monoclonal antibody reactive with IESCA) or another antibody capable (able to) recognize IESCA, and sera from individuals suspected of having AISNHL.

Furthermore, the present invention also contemplates using the above-named antigen and compositions in screening assays for AISNHL. The present invention contemplates a variety of methods of testing patients suspected of having AISNHL. The present invention is not limited by the particular method of screening. In one embodiment, guinea pig (or other suitable animal) organ of Corti (or other suitable tissue) will be exposed to patient's sera followed by immunofluorescent staining by methods known to those in the field. A positive result comprises a distinctive staining pattern of “wine glass” shapes on the organ of Corti (or other distinguishable pattern depending on the tissue used). Staining with MAb KHRI-3, or other suitable antibody, may serve as a positive control. The invention is not limited by the tissue used or the animal from which it was derived so long as when the tissue is stained with antibody that recognizes IESCA, the staining is distinctive. The invention is not limited by the antibody used as a positive control, so long as the antibody is specific for IESCA or an epitope of IESCA.

A second method contemplates acquiring substantially purified IESCA from source tissue and determining if sera from a patient suspected of having AISNHL will react with the antigen. The present invention is not limited by the particular method of screening. A particular embodiment comprises 1) making tissue extracts from guinea pig organ of Corti, followed by 2) immunoprecipitation of IESCA with monoclonal antibody KHRI-3, or other suitable antibody, and 3) Western blotting precipitated IESCA with patients' sera. A positive result comprises recognition of the precipitated IESCA with the patient's sera. Staining with MAb KHRI-3, or other suitable antibody, may serve as a positive control. The invention is not limited by the tissue used to acquire IESCA or the animal from which it was derived so long as the isolated protein stains with antibody that recognizes IESCA. The invention is not limited by the antibody used to precipitate the IESCA, or used as a positive control, so long as the antibody is specific for IESCA or an epitope of IESCA (which can be confirmed in competition assays or preclearing assays with MAb KHRI-3).

The invention also contemplates drug screens for compounds that are agonistic or antagonistic for binding to IESCA and anti-IESCA. In one embodiment, compounds suspected of binding IESCA would be mixed with IESCA, under conditions that permit the binding of the compound to IESCA, followed by washing and then the addition of anti-IESCA. Binding of anti-IESCA to IESCA would be detected by using assays known to those practiced in the art. Increases or deceases in binding as compared to controls would be used to determine if the suspected compound was agonistic or antagonistic to the binding of IESCA and anti-IESCA.

In another embodiment, a compound suspected of binding anti-IESCA, but not IESCA, is mixed with anti-IESCA, under conditions that permit the binding of the compound to IESCA, followed by washing and then the addition of IESCA. The binding of IESCA to anti-IESCA would be detected by assays known to those practiced in the art. Increase or decreases in binding as compared to controls would be used to determine if the suspected compound was agonistic or antagonistic to the binding of IESCA to anti-IESCA. Both screening assays described above are adaptable to high throughput assay formats, for example by utilizing microtiter plates and automatic plate readers.

The invention contemplates methods for screening for intra- and inter- specific homologs of IESCA, one method comprising (for example): a) providing in any order: i) extracts from cell suspected of containing said homolog, ii) antibodies reactive to IESCA and specific for at least a portion of the peptide or glycoconjugate IESCA; and b) mixing the said antibody with said extract under conditions such that said homolog is detected.

The present invention contemplates a substantially purified glycoprotein from the inner-ear organ of Corti reactive with KHRI-3 monoclonal antibody.

The present invention also contemplates a composition comprising guinea pig organ of Corti and sera from patients suspected of having AISNHL.

The present invention further contemplates a composition comprising extracts from guinea pig organ of Corti, the KHRI-3 monoclonal antibody and sera from patients suspected of having AISNHL.

The present invention further contemplates a method to detect antibodies in a patient's sera, comprising: a) exposing guinea pig organ of Corti to patient's sera, said sera comprising antibodies; and b) visualizing bound antibody.

The present invention further contemplates a method to detect antibodies in a patient's sera comprising: a) preparing an extract from guinea pig organ of Corti; b) contacting the extract with KRHI-3 monoclonal antibody; and c) isolating an antigen-antibody complex.

The present invention also contemplates the method of claim 5, further comprising detecting the antigen with sera from patients suspected of having AISNHL.

The present invention further contemplates a method to detect agonist and antagonist binding to IESCA. Such methods comprising: a) a compound suspected of binding IESCA; b) contacting IESCA with said compound; c) detecting increases or decreases in binding of IESCA to anti-IESCA as compared to controls.

The present invention further contemplates a method to detect agonist and antagonist binding to anti-IESCA comprising: a) a compound suspected of binding anti-IESCA; b) contacting anti-IESCA with said compound; c) detecting increases or decreases in binding of anti-IESCA to IESCA as compared to controls.

The present invention further contemplates a method to screen for homologs of IESCA comprising: a) extracts from cells suspected of containing said homolog; b) contacting the extract with anti-IESCA; c) detecting said homolog by techniques known to those practiced in the art.

### DESCRIPTION OF THE FIGURES

FIG. 1 shows an exemplary amino acid sequence of an N-terminal portion of IESCA (SEQ ID NO:1).

FIG. 2 shows an exemplary amino acid sequence of an N-terminal portion of IESCA (SEQ ID NO:2).

FIG. 3 shows a graphical representation of protein G purification of KHRI-3 monoclonal antibody.

FIG. 4 shows an SDS-PAGE gel of purification of IESCA using a KHRI-3 antibody affinity column.

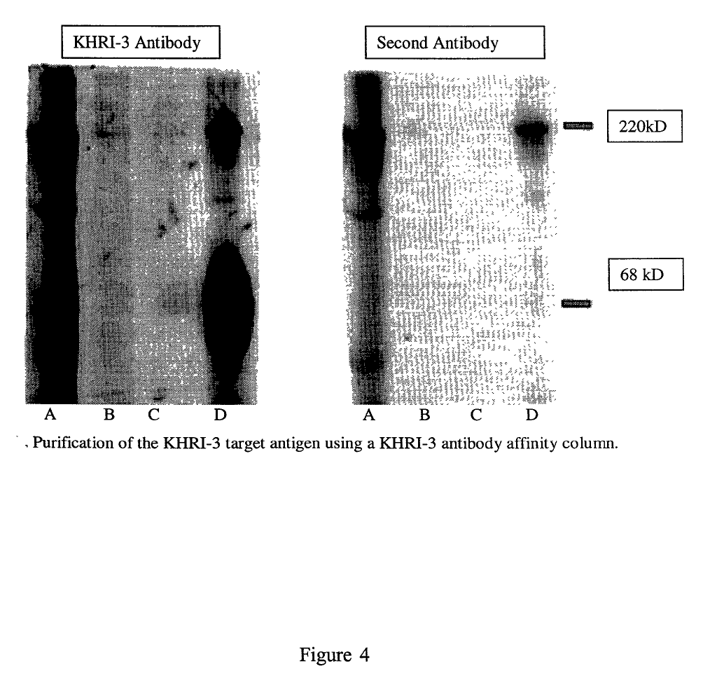

FIG. 5 shows a non-reducing and reducing PAGE of purified IESCA.

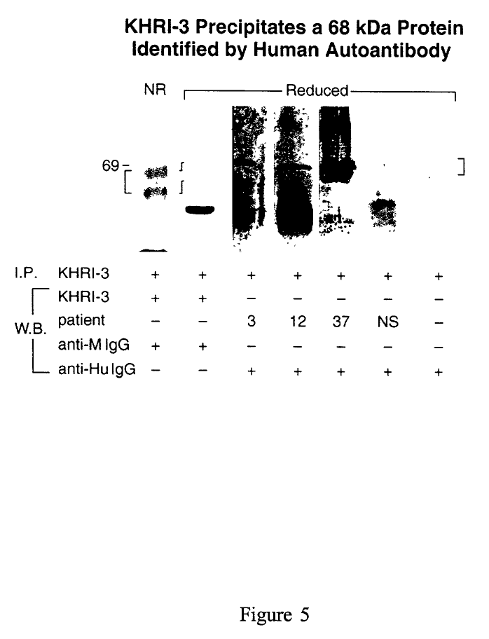

### DEFINITIONS

To facilitate understanding of the invention, a number of terms are defined below.

The term “homology” when used in relation to proteins refers to a degree of complementarity. There may be partial homology or complete homology (i.e., identity). A partially complementary sequence is one that at least partially inhibits a completely complementary sequence performing its function (e.g., enzymatic, binding, etc) in vivo or in vitro and is referred to using the functional term “substantially homologous.” The inhibition function of the completely complementary sequence may be examined using an enzymatic assay, a binding assay or other assay designed to measure the particular function of the completely complementary protein. A substantially homologous sequence or probe will compete for and inhibit the function (e.g., the binding or enzymatic function) of a sequence which is completely homologous to a target under conditions of low stringency. This is not to say that conditions of low stringency are such that non-specific interaction is permitted; low stringency conditions require that the interaction of the sequence with its substrate be a specific (i.e., selective) interaction. The absence of non-specific binding may be tested by the use of a second target which lacks even a partial degree of complementarity (e.g., less than about 30% identity); in the absence of non-specific interaction the probe will not react to the second non-complementary target.

As used herein, the term “purified” or “to purify” refers to the removal of contaminants from a sample. The present invention contemplates purified compositions (discussed above).

As used herein, the term “substantially purified” refers to the removal of a portion of the contaminants of a sample to the extent that the substance of interest is recognizable by techniques known to those skilled in the art.

As used herein the term “portion” when in reference to a protein (as in “a portion of a given protein”) refers to fragments of that protein. The fragments may range in size from four amino acid residues to the entire amino acid sequence minus one amino acid.

“Inner-ear Supporting Cells Antigen (IESCA)” shall be defined as molecules (proteins, glycoproteins, lipoproteins or other molecules reactive with antibodies), or portions thereof, that are localized with high specificity to the supporting cells of the inner ear and are reactive with KHRI-3 MAb. In the context of this patent application the following shall apply: Expression of these molecules in other tissues does not exclude them from being IESCA. Likewise, absence of these molecules from the inner ear supporting cells in certain circumstances (such as disease) does not exclude them from being IESCA. Furthermore, variation in molecular weight of IESCA from that disclosed here, so long as they are reactive with KHRI-3 MAb, does not exclude them from being IESCA.

“Staining” shall be defined as any number of processes known to those in the field that are used to better visualize, distinguish or identify a specific component(s) and/or feature(s) of a cell or cells.

“Immunofluorescence” is a staining technique used to identify, mark, label, visualize or make readily apparent by procedures known to those practiced in the art, where a ligand (usually an antibody) is bound to a receptor (usually an antigen) and such ligand, if an antibody, is conjugated to a fluorescent molecule, or the ligand is then bound by an antibody specific for the ligand, and said antibody is conjugated to a fluorescent molecule, where said fluorescent molecule can be visualized with the appropriate instrument (e.g., a fluorescent microscope).

“Autoimmune (autoimmunity)” shall be defined as a condition where antibodies recognize self-antigens as foreign and thereby initiate an immune response to cells, tissues or organs often causing the establishment and continuation of a disease state.

“Sensorineural hearing loss (SNHL)” shall be defined as a disease characterized by progressive unilateral or bilateral deafness resulting from damage to sensory cells or nearves or other components of the inner ear necessary for processing sounds, that, in its incipient stages, may fluctuate or become sudden and profound.

“Autoimmune sensorineural hearing loss (AISNHL)” shall be defined as SNHL whose etiology includes the generation of autoantibodies directed against inner ear antigenic epitopes.

“Antibody” shall be defined as a glycoprotein produced by B cells that binds with high specificity to the agent (usually, but not always, a peptide), or a structurally similar agent, that generated its production. Antibodies may be produced by any of the known methodologies [Current Protocols in Immunology (1998) John Wiley and Sons, Inc., N.Y.] and may be either polyclonal or monoclonal.

“Antigen” shall be defined as a protein, glycoprotein, lipoprotein, lipid or other substance that is reactive with an antibody specific for a portion of the molecule.

The terms “immunoprecipitate”, “immunoprecipitated”, “immunoprecipitation” and “affinity purification” shall refer to the use of an antibody to separate its antigen or a portion thereof from a mixture of other molecules.

“Morphology” shall be defined as the visual appearance of a cell or organism when viewed with the eye, a light microscope, a confocal microscope or an electronmicroscope, as appropriate.

“Patient” shall be defined as a human or other animal, such as a guinea pig or mouse and the like, capable of having AISNHL, either naturally occurring or induced.

“Inner ear antigens” shall be defined as antigens from the inner ear that are reactive with autoantibodies associated with AISNHL.

### GENERAL DESCRIPTION OF THE INVENTION

The present invention pertains to a substantially purified novel antigen (IESCA) and to compounds and methods for the screening of sera for antibodies reactive to the antigen. The presence of these antibodies in the patient's sera is predictive of a patient's potential to respond to steroid treatment for AISNHL. Additionally, the invention comprises methods for the detection of compounds that are agonistic and/or antagonistic for IESCA binding and/or anti-IESCA binding, as well as methods for the identification of intra- and inter-specific homologs of IESCA.

A. Autoimmune Sensorineural Hearing Loss (AISNHL)

Autoimmunity as a cause of sensorineural hearing loss has yet to be completely confirmed or characterized. However, the evidence of antibody to inner-ear antigens in sera from patients with the clinical diagnosis of autoimmune sensorineural hearing loss (AISNHL) and experimental models strongly support this possibility (Harris, “Immunology Of The Inner Ear: Response Of The Inner Ear To Antigen Challenge,” Otolaryngol. Head Neck Surg., 91:18-23, 1983; Yoo et al., “Factors Influencing Collagen-Induced Autoimmune Ear Disease,” Am. J. Otolaryngol, 6:209-216, 1983; Harris, “Experimental Autoimmune Sensorineural Hearing Loss,” Laryngoscope, 97:63-76, 1987; Harris and Sharp, “Inner Ear Autoantibodies In Patients With Rapidly Progressive Sensorineural Hearing Loss,” Laryngoscope, 100:516-524, 1990; Orozco et al., “Experimental Model Of Immune-Mediated Hearing Loss Using Cross-Species Immunization,” Laryngoscope, 100:941-947, 1990; Cruz et al., “Autoimmune Sensorineural Gearing Loss: A Preliminary Experimental Study,” Am. J. Otol., 11:342-346, 1990; McCabe, “Autoimmune Inner Ear Disease: Results Of Therapy,” Adv. Otorhinolaryngol, 46:78-81, 1991; Kusakari et al., “MLR/MP-lpr/lpr Mouse As A Model Of Immune-Induced Sensorineural Hearing Loss,” Ann. Otol. Rhinol Laryngol., 101:82-86, 1992; Tago et al., “Cochlear And Renal Pathology In The Autoimmune Strain Mouse,” Ann. Otol. Rhinol. Laryngol, 157(Suppl.):87-91, 1992; Wong et al., “Cochlear IgG In The C3H/lpr Autoimmune Mouse Strain,” Hear. Res., 59:93-100, 1992; Sone et al., “A Substrain Of NZB Mouse As An Animal Model Of Autoimmune Inner Ear Disease,” Hear. Res., 83:26-36, 1994; Moscicki et al., “Serum Antibody To Inner Ear Proteins In Patients With Progressive Hearing Loss: Correlation With Disease Activity And Response To Corticosteroid Treatment,” JAMA, 272:611-616, 1994; Harris and Ryan, “Fundamental Immune Mechanisms Of The Brain And Inner Ear,” Otolaryngol. Head Neck Surg., 112:639-653, 1995; Billings et al., “Evidence Linking The 68 Kilodalton Antigen Identified In Progressive Sensorineural Hearing Loss Patient Sera With Heat Shock Protein 70,” Ann. Otolol. Rhinol. Larngol., 104:181-188, 1995; Gottschlich et al., “Assessment Of Serum Antibodies In Patients With rapidly Progressive Sensorineural Hearing Loss And Meniere's Disease,” Laryngoscope, 105:1347-1352, 1995; Cao et al., “Detection Of Inner Ear Disease Autoantibodies By Immunoblotting,” Mol. Cell Biochem., 146:157-163, 1995; Nair et al., “Monoclonal Antibody Induced Hearing Loss,” Hear. Res., 83:101-113, 1995; Cao et al., “Guinea Pig Inner Ear Antigens: Extraction And Application To The Study Of Human Autoimmune Inner Ear Disease,” Laryngoscope, 106:207-212, 1996). In this regard, the importance of identifying antigens specific for AISNHL for the establishment of accurate method for diagnoses becomes critical.

In 1987 Harris (Harris, “Experimental Autoimmune Sensorineural Hearing Loss,” Laryngoscope, 97:63-76, 1987) demonstrated hearing loss and inner-ear lesions in guinea pigs immunized with bovine inner-ear extract. Subsequently, Orosco et al. (Orozco et al., “Experimental Model Of Immune-Mediated Hearing Loss Using Cross-Species Immunization,” Laryngoscope, 100:941-947) immunized mice and guinea pigs with chick and guinea pig inner-ear tissues and found that the animals developed transient hearing loss and serum antibodies to hair cell stereocilia.

The present inventors followed these experiments with the development of monoclonal antibodies to inner-ear antigens by immunizing mice with chick and guinea pig inner-ear tissue (Zajic et al., “Monoclonal Antibodies To Inner Ear Antigens: I. Antigens Expressed By Supporting Cells In Guinea Pig Cochlea,” Hear. Res., 52:59-72, 1991; Ptok et al., “Monoclonal Antibodies To Inner Ear Antigens: II. Antigens Expressed In Sensory Cell Stereocilia,” Hear. Res., 57:79-90, 1991). Two classes of monoclonal antibodies (MAb) to inner-ear antigens were characterized. One class (KHRI-5 and KHRI-6) stains stereocilia (Ptok et al., “Monoclonal Antibodies To Inner Ear Antigens: II. Antigens Expressed In Sensory Cell Stereocilia,” Hear. Res., 57:79-90, 1991). The other class is represented by MAb KHRI-3, which binds to inner ear supporting cells in a characteristic punctate “tacked wine glass” staining pattern (Zajic et al., “Monoclonal Antibodies To Inner Ear Antigens: I. Antigens Expressed By Supporting Cells In Guinea Pig Cochlea,” Hear. Res., 52:59-72, 1991). It also identifies a protein in Western blots of inner-ear extracts (Nair et al., “Monoclonal Antibody Induced Hearing Loss,” Hear. Res., 83:101-113, 1995).

B. Animal Models For The Support Of AISNHL

In vivo studies showed that mice carrying the KHRI-3 hybridoma develop high-frequency hearing loss (Nair et al., “Monoclonal Antibody Induced Hearing Loss,” Hear. Res., 83:101-113, 1995; Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995). The hearing loss is associated with high circulating KHRI-3 antibody titers and loss of outer hair cells in the basal turn of the cochlea, the region that encodes high-frequency sounds. More recent studies using in vivo infusion of KHRI-3 antibody directly into the guinea pig cochlea have shown that KHRI-3 binds to supporting cells in vivo, and that animals receiving this antibody, but not an isotype-matched IgG1 myeloma protein, develop hearing loss (Nair et al., “In vivo Binding And Hearing Loss After Intracochlear Infusion Of KHRI-3 Antibody,” Hear. Res., 107:93-101, 1997). These experimental studies provide strong evidence for antibody-mediated disruption of hearing.

C. AISNHL In Humans

Support for antibody-mediated hearing loss in humans also has accumulated. Harris and his colleagues demonstrated that serum antibodies from patients with AISNHL bind to a 68 kD protein in bovine inner-ear tissue extracts (Harris and Sharp, “Inner Ear Autoantibodies In Patients With Rapidly Progressive Sensorineural Hearing Loss,” Laryngoscope, 100:516-524, 1990; Harris and Ryan, “Fundamental Immune Mechanisms Of The Brain And Inner Ear,” Otolaryngol. Head Neck Surg., 112:639-653, 1995; Billings et al., “Evidence Linking The 68 Kilodalton Antigen Identified In Progressive Sensorineural Hearing Loss Patient Sera With Heat Shock Protein 70,” Ann. Otolol. Rhinol. Larngol., 104:181-188, 1995; Gottschlich et al., “Assessment Of Serum Antibodies In Patients With Rapidly Progressive Sensorineural Hearing Loss And Meniere's Disease,” Laryngoscope, 105:1347-1352, 1995). In a recent report, the cumulative data were summarized (Harris and Ryan, “Fundamental Immune Mechanisms Of The Brain And Inner Ear,” Otolaryngol. Head Neck Surg., 112:639-653, 1995). Of 279 patients from patients with rapidly progressive sensorineural hearing loss, 32% were positive for the 68 kD antigen on Western blots (Harris and Ryan, “Fundamental Immune Mechanisms Of The Brain And Inner Ear,” Otolaryngol. Head Neck Surg., 112:639-653, 1995). Hughes et al (Hughes et al., “Laboratory Diagnosis Of Immune Inner Ear Disease,” Am. J. Otology., 15:198-202, 1994) reported finding antibodies to a 68 kD antigen in 86% of patients with what they termed idiopathic, progressive, bilateral sensorineural hearing loss (IPBSNHL), whereas Moscicki et al. (Moscicki et al., “Serum Antibody To Inner Ear Proteins In Patients With Progressive Hearing Loss: Correlation With Disease Activity And Response To Corticosteroid Treatment,” JAMA, 272:611-616, 1994) found this type of antibody activity in 58% of patients. These groups also found that the presence of antibody predicted a clinical response to treatment with steroids.

D. Identification Of The 68 kD Protein As HSP-70

The nature of the target antigen of human AISNHL autoimmune sera has been suggested to be heat shock protein (HSP) 70. Harris (Billings et al., “Evidence Linking The 68 Kilodalton Antigen Identified In Progressive Sensorineural Hearing Loss Patient Sera With Heat Shock Protein 70,” Ann. Otolol. Rhinol. Larngol., 104:181-188, 1995) demonstrated this using ion exchange chromatography, adenosine triphosphate affinity chromatography, and one- and two-dimensional electrophoresis. Additionally, immunoblotting with AISNHL patient sera showed dramatically increased expression of the 68 kD antigen by bovine kidney cells following heat shock in culture. HSP-70 is a ubiquitous protein that is not specific for the inner-ear. However, reactivity with stress proteins of various classes has been reported in a number of autoimmune diseases as well as non-autoimmune disease states. When cells or organisms are placed under stress, synthesis of HSPs increases presumably to protect critical proteins from denaturation. Thus, antibodies to HSPs may be produced as a result of release of cellular contents of damaged cells under the same stress that leads to formation of antibodies to autoimmune specific antigens. In fact, surveys of patients with other autoimmune diseases and patients with some types of infection also frequently have antibodies to heat shock proteins. In spite of the lack of specificity, HSP-70 is being used in some studies, including a national clinical trial sponsored by the NIH (www.entnet.org/aiedtrail.html), as a target antigen for assessing AISNHL.

E. IESCA And HSP-70 Are Different Proteins

The present inventors have conducted experiments that establish IESCA and HSP-70, in spite of being similar molecular weight, are different proteins. First, antigen purified from patient tissue or guinea pig inner ear with the IESCA specific MAb KHRI-3 does not react with antibodies to HSP-70. Similarly, protein precipitated by HSP-70 antibody does not react with the KHRI-3 MAb. Likewise, anti-HSP-70 does not stain guinea pig organ of Corti supporting cells, as does MAb KHRI-3. These differences have led us to conclude that IESCA and HSP-70 are not similar.

F. Inner-ear Supporting Cell Antigen As Screening Agent For AISNHL

The presence of IESCA antibodies in patients sera has been shown to have a high correlation with the probable response of a patient to corticosteroid treatment for AISNHL. Therefore, a diagnostic screen based on the detection of anti-IESCA antibodies would identify these patients while protecting those that do not express anti-IESCA from unnecessary treatment and any associated side effects. Additionally, the identification of compounds that are agonistic and/or antagonistic to IESCA binding and/or anti-IESCA binding may be advantageous in determining methodologies for the treatment of AISNHL.

### DETAILED DESCRIPTION OF PREFERRED EMBODIMENTS

Generally, the nomenclature used hereafter and the laboratory procedures in cell culture, molecular genetics, and nucleic acid chemistry and hybridization described below are those well known and commonly employed in the art. Standard techniques are used for tissue extractions, electrophoresis, recombinant nucleic acid methods, polynucleotide synthesis, and microbial culture and transformation (e.g., electroporation, lipofection). Generally enzymatic reactions and purification steps are performed according to the manufacturer's specifications. The techniques and procedures are generally performed according to conventional methods in the art and various general references. [See, generally, Sambrook et al., Molecular Cloning: A Laboratory Manual, 2d ed. (1989) Cold Spring Harbor Laboratory Press, Cold Spring Harbor, N.Y., and Current Protocols in Molecular Biology (1996) John Wiley and Sons, Inc., N.Y.].

The nomenclature used hereafter and the laboratory procedures in immunology are those well known and commonly employed in the art. Standard techniques are used for antibody generation and purification, immunoprecipitation, Western blotting, staining and immunofluorescence. The techniques and procedures are generally performed according to conventional methods in the art and various general references. [See, generally, Current Protocols in Immunology (1998) John Wiley and Sons, Inc., N.Y.].

The current invention comprises the identification and substantial purification of a novel protein from guinea pig organ of Corti. The current invention also contemplates compositions and methods for the detection of AISNHL in a patient. Additionally, a kit containing reagents to assay for an antibody associated with AISNHL in a patient is disclosed and claimed by this invention. Furthermore, drug screens for compounds that are agonistic or antagonistic for IESCA or anti-IESCA binding are claimed by this invention. Likewise, screens for homologs of IESCA are claimed by this invention. The invention represents a major improvement over existing tests for AISNHL which identify antibodies to a universally distributed substance (HSP-70) that is not unique to the inner ear and has never been linked to hearing loss.

1. Screens To Identify IESCA Antibodies In Patient's Sera

A. Specific Binding Of IESCA Antibodies To Inner Ear Supporting Cells

IESCA is preferentially expressed on the surface of guinea pig organ of Corti. It is believed that an autoimmune response to this antigen may be responsible for the onset and continuation of AISNHL. IESCA is a conserved antigen that is recognized immunologically across species. Patients that express antibodies to IESCA could then be treated with conventional therapy which includes that use of immune modulating drugs such as corticosteroids, cyclophosphamide, methotrexate and cyclosporin A. In one embodiment, serum from patients suspected of having AISNHL will be used to stain guinea pig organ of Corti inner-ear supporting cells. The organ of Corti will be excised from guinea pig and exposed to patient sera. After washing, the organ of Corti will be exposed to a human IgG specific, second step fluorescent conjugated antibody. A positive reaction will be the distinctive “wine glass” staining pattern. Staining with the anti-IESCA MAb antibody KHRI-3 will serve as a positive control. In other embodiments, organ of Corti from other animals may be used as well as different anti-human IgG second step antibodies. Additionally, other control antibodies may be used as they become available.

B. Specific Binding Of IESCA Antibodies To Immunoprecipitated Or Affinity Purified IESCA

In another embodiment, patients will be tested for the presence of anti-IESCA antibodies following the immunoprecipitation or antibody affinity purification of IESCA with KRHI-3 MAb from guinea pig organ of Corti. Patients sera is then used to detect the precipitated or purified IESCA by Western blot. A positive response is the staining of a band at about 68-70 kD. Staining with the anti-IESCA MAb antibody KHRI-3 will serve as a positive control. In other embodiments, IESCA precipitated from other animals may be used. Additionally, other IESCA reactive antibodies may be used for immunoprecipitation or as control antibodies as they become available. In further embodiments, precipitated IESCA or IESCA or IESCA fragments or components produced via molecular biological methods may be used in microtiter plate assays (e.g., ELISA assays) or similar.

2. Screens To Identify Compounds That Are Antagonistic Or Agonistic For IESCA Binding

In some embodiments, IESCA (and in particular, fragments of IESCA) are useful in drug screening assays designed to identify drugs that interfere with the specific binding of anti-IESCA to IESCA, thereby blocking the degeneration of the antigen and progression of autoimmune sensorineural hearing loss.

In particular embodiments, the invention provides isolated IESCA or IESCA polypeptide fragments and/or carbohydrate or other protein modifications. The claimed polypeptide and fragments find particular use in screening assays for agents or lead compounds for agents useful in the diagnosis, prognosis or treatment of disease, particularly disease associated with AISNHL. One such assay involves forming mixtures of 1) IESCA (or fragments thereof) and 2) anti-IESCA (which may or may nor be KHRI-3 MAb), in the presence of 3) a prospective drug candidate. The mixtures are made under conditions that permit the binding of IESCA with anti-IESCA antibody and the mixtures are then analyzed for the presence of such binding. A difference in such binding in the presence of a drug candidate indicates that the agent is capable of modulating the binding of IESCA (or fragments thereof) to anti-IESCA antibody. The assays of the present invention provide for the facile high-throughput screening of compounds suspected to be able to inhibit such binding (e.g., compound libraries, peptide libraries, and the like) to identify potential drug candidates.

3. Screens To Identify Compounds That Are Antagonistic Or Agonistic For Anti-IESCA Binding

In another embodiment, anti-IESCA antibodies (or fragments thereof), may be used to screen for compounds that specifically bind to anti-IESCA antibodies and thereby interfere with the binding of anti-IESCA antibodies to IESCA, without binding IESCA. In one embodiment the invention, IESCA (or fragments thereof), will be used to compete against prospective drug candidates in a competition assay. Additionally, it may be used as a positive control.

In particular embodiments, the invention utilizes isolated anti-IESCA antibody or anti-IESCA antibody fragments. The antibody and fragments find particular use in screening assays for agents or lead compounds for agents useful in the diagnosis, prognosis or treatment of disease, particularly disease associated with AISNHL. One such assay involves forming mixtures of 1) anti-IESCA antibodies (or fragments thereof) and 2) a prospective drug candidate, and, finally, 3) IESCA (or fragments thereof). The mixtures are made under conditions that permit the binding of the prospective drug candidate with anti-IESCA antibody, followed by the addition of IESCA (or fragments thereof). The mixtures are then analyzed for the presence of such binding of anti-IESCA antibody to IESCA (or fractions thereof). A difference in such binding in the presence of a drug candidate indicates that the agent is capable of modulating the binding of IESCA (or fragments thereof) to anti-IESCA antibody. The assays of the present invention provide for the facile high-throughput screening of compounds suspected to be able to inhibit such binding (e.g., compound libraries, peptide libraries, carbohydrates, and the like) to identify potential drug candidates.

4. Screens To Identify IESCA Homologs

Standard molecular biological and immunological techniques can be used to identify IESCA homologs. For example, the present invention contemplates a number of approaches including, but not limited to, immunoprecipitation and affinity purification of cell and tissue extracts and immunoscreening of proteins and glycoproteins translated from DNA and RNA library stocks. Furthermore, hybridization screens of RNA and DNA library stocks could be accomplished using RNA and DNA sequences reverse engineered from isolated IESCA protein.

### EXPERIMENTAL

The following examples are intended to illustrate, but not limit, the present invention.

In the preliminary studies, the present inventors used guinea pig inner ear tissue as the substrate for detection of inner ear reactive antibodies in sera from patients with AISNHL. We made this choice because we have extensive experience with this model (Zajic et al., “Monoclonal Antibodies To Inner Ear Antigens, I. Antigens Expressed By Supporting Cells Of The Guinea Pig Cochlea,” Hearing Res., 52:59-72, 1991; Ptok et al., “Monoclonal Antibodies To Inner Ear Antigens, II. Antigens Expressed In Sensory Cell Stereocilia,” Hearing Res., 57:79-90, 1991; Nair et al., “Monoclonal Antibody Induced Hearing Loss,” Hearing Res., 83:101-113, 1995; Nair et al., “In vivo Binding And Hearing Loss After Intracochlear Infusion Of KHRI-3 Antibody,” Hearing Res., 107:93-101, 1997; Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995), we can control precisely the preparation of the inner ear extract, and much previous work has indicated that inner ear reactive antibodies bind to phylogenetically conserved antigens (Harris, “Immunologic Mechanisms In Disorders Of The Inner Ear,” In: 2nd edition Otolaryngology Head and Neck Surgery Vol. IV: Ear and Cranial Base, Cummings et al. (eds.) Mosby Yearbook, St. Louis, Mo., pp. 2926-2942, 1993; Harris and Ryan, “Immunobiology Of The Inner Ear,” Am. J. Otolaryngol., 1984). In our initial reports we found that 50-60% of patients clinically assessed as having AISNHL have antibodies that bind to a 68-70 kD protein in guinea pig inner ear extracts. The same sera also stain supporting cells in the organ of Corti with a pattern like that of the KHRI-3 monoclonal antibody. This is the first demonstration that Western blot positive sera from humans with rapidly progressive hearing loss have a reproducible binding pattern in the inner ear (Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995). Also, we showed that a 68-70 kD inner ear protein immunoprecipitated by KHRI-3 is strongly stained by antibodies in AISNHL patients' sera, but not sera from normal control donors. Furthermore, human inner ear tissue, but not blood cells from the same donor, absorbs the human serum antibody reactivity to guinea pig inner ear substrate indicating that the antibodies define a phylogenetically conserved protein expressed in human and guinea pig ears. Our data strongly suggest that the KHRI-3 monoclonal antibody and human sera from patients with the provisional diagnosis of AISNHL bind to the same antigen. We postulate that human antibodies against this antigen are likely to be a cause of hearing loss. We base this on experimental studies showing that the KHRI-3 antibody can bind to supporting cells in vivo, results in loss of sensory cells in the organ of Corti and the development of significant hearing loss. Additionally, patients with antibody to the supporting cell antigen were statistically significantly more likely to have improvement in hearing after steroid therapy than those without such antibody (p=0.008, Fisher's exact test, Relative Risk 2.4, unpublished data). In contrast there was no difference in the rate of response in patients who were either Western blot positive or negative (p=0.79). In fact, of those who responded to steroids 88% were positive for the supporting cell antibody and only 12.1% were negative by this assay. The likelihood of response to steroid therapy is more than two times greater in those with antibody to supporting cells than those without such antibody.

### EXAMPLE I

### Isolation Of KHRI-3 MAb

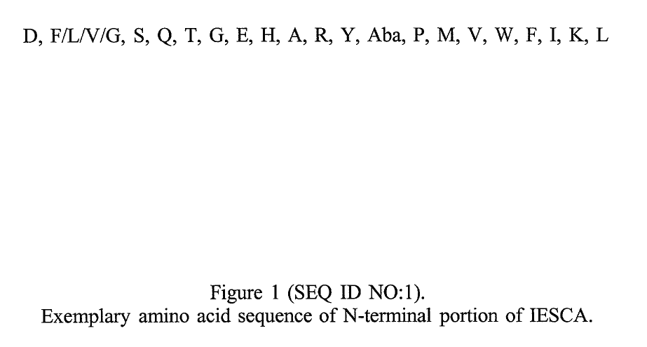

Hybridoma cells were cultured in a CELLMAX (CELLMAX® Artificial Capillary System, Cellco, Inc., Germantown, Md.) bioreactor. The bioreactor was inoculated with KHRI-3 hybridoma cells. The serum-free, antibiotic-free medium was changeded on lactic acid production to maintain optimal cell growth and antibody production. Once the system was operating at optimal conditions, 45 ml of fluid containing antibody and excess cells was harvested every second day from the extracapillary space. The IgG1 antibody binding capacity of a protein G affinity column was determined. An amount of bioreactor supernatant calculated to contain that quantity of antibody was loaded and the effluent was monitored for absorbance at A₂₈₀. After all of the sample was loaded, the column was washed with binding buffer. The elution buffer was applied when the protein content of the effluent reached zero absorbance and the second peak was collected in three fractions corresponding to the center and borders of the peak (FIG. 1). Protein and IgG content of each fraction was assessed using Bradford assays, IgG ELISA and SDS-PAGE followed by Western blotting with anti-Ig antibody. Eight column runs were pooled to yield 80.5 mg of antibody.

### EXAMPLE II

### Purification Of The KHRI-3 Inner Ear Target Antigen By Affinity Chromatography

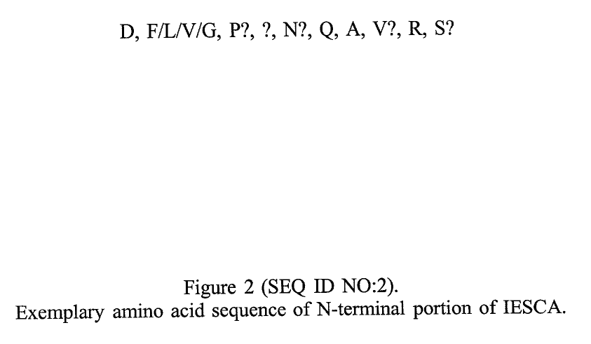

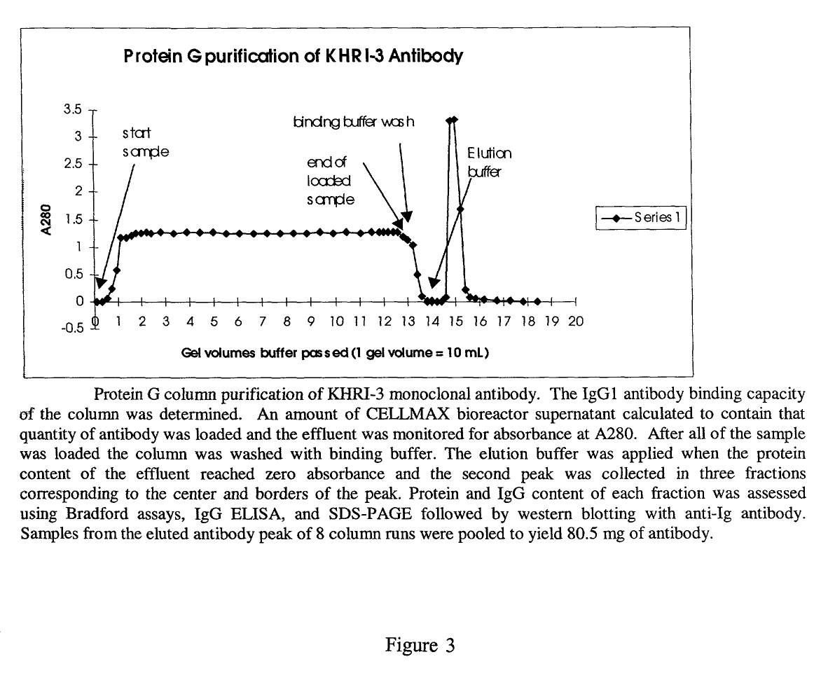

KHRI-3 MAb, affinity purified on protein G sepharose, was covalently linked to sepharose beads and used to affinity purify IESCA. Inner ear extract was passed onto the column after washing the column and was rinsed extensively with binding buffer. The samples coming off the column were monitored for protein using absorbance at 280 nm. When no protein was detected in the rinse buffer, the bound material was eluted with glycine-HCL (pH 2.0) buffer. The fractions were collected in twenty 1 ml fractions. Each fraction was monitored for absorbance at 280 nm. Pooled fractions (1-5, 6-10, 11-15 and 16-20) were concentrated to 100 μl, electrophoresed on a 7% SDS-PAGE gel, transferred to nitrocellulose and Western blotted using chemiluminescence. Fraction pool 16-20 alone contained KHRI-3 reactive protein as determined by Western blot (FIG. 2). When the SDS-PAGE gel was run under reducing conditions a strong doublet was detected at 68 kD and 70 kD (FIG. 3). Under non-reducing conditions a doublet was resolved at 65 kD and 68 kD. Stripping and reprobing the blot with anti-IgG antibody showed that the band was not due to free IgG leaching from the column.

### EXAMPLE III

### Autoimmune Sensory Neural Hearing Loss Patients

Patients with sudden onset hearing loss, fluctuating hearing and rapidly progressive unilateral or bilateral hearing loss were considered possible autoimmune patients. Sudden onset cases were included after review of the histories of several of our bilateral progressive cases showed that these patients often reported an initial event of sudden hearing loss in one ear that subsequently progressed to involve the opposite ear. We included this group since they may represent the acute stage of early disease. Hearing loss was defined as greater than 30 dB at any frequency or less than 85% speech discrimination in either one (unilateral) or both ears (bilateral). If the hearing loss developed within a 24 hour period and did not progress subsequently, the patient was considered to have sudden hearing loss. Either unilateral or bilateral hearing loss with greater than 10 dB progression within three months was classified as rapidly progressive (Moscicki et al., “Serum Antibody To Inner Ear Proteins In Patients With Pprogressive Hearing Loss: Correlation With Disease Activity And Response To Corticosteroid Treatment,” JAMA, 272:611-616, 1994). The initial analysis of 91 patients has been published (Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995). Of the 91 patients, seventeen did not fit in these categories were not included in the analysis. Four patients with vertigo and 1 with tinnitus, were excluded because of insufficient hearing loss to fit the criteria. Seven patients were classified as slowly progressive hearing loss (i.e., less than a 10 dB progression in three months); four patients were not well classified and more data has been requested; and one patient had stable hearing loss leaving 74 in the group.

### EXAMPLE IV

### Western Blot Assays

Sera from the remaining 74 patients with rapidly progressive or sudden onset hearing loss were evaluated for reactivity with guinea pig inner ear antigens using Western blot (73 patients). The breakdown of the patients by type of hearing loss and serological result is shown in Table 1. Fifty-four patients (36 bilateral and 18 unilateral) had rapidly progressive hearing loss. Twenty patients (2 bilateral and 18 unilateral) had sudden hearing loss. Of the 54 patients with rapidly progressive hearing loss tested by Western blot, 28 (52%) stained a 68-70 kD protein. Similarly, one-half of the sudden hearing loss patients, 9/19(47%), were positive. Of the 54 patients with rapidly progressive hearing loss, 50% (18/36) with bilateral involvement and 56% (10/18) with unilateral involvement were Western blot positive (Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995).

TABLE 1
Western Blot Results By Type Of Hearing Loss

| | Type Of Hearing Loss | | |
|---|---|---|---|
| | Rapidly | | |
| | Progressing | Sudden | Total |
| Number of Patients | 54 | 20 | 74 |
| Western Blot: No. Positive/ | 28/54/52% | 9/19/47% | 37/73/51% |
| No. Tested/% Positive | | | |

### EXAMPLE V

### Immunofluorescence Assays

To determine the location of the inner ear antigen, we tested Western blot positive sera, Western blot negative sera, normal donor sera, and sera from previously untested patients for immunofluorescence staining on surface preparations of guinea pig organ of Corti. Including the previously reported cases a total of 166 AISNHL patients have now enrolled in the study. Sera from 158 have been studied for antibody to a 68 kD inner ear protein by Western blot and 143 have been tested by immunofluorescence on organ of Corti surface preparations. Many sera stained supporting cells. The staining was distributed in punctate clusters over the surfaces of the supporting cells, including the surface of the pillar cells, the phalangeal processes of the outer pillar cells, and the phalangeal processes of the Deiters' cells. There was a very strong relationship between sera that were Western blot positive and those that stained supporting cells as shown in Table 2.

TABLE 2
Results For Sera From Suspected Autoimmune Hearing Loss Patients
By Both Assays

Immunofluorescence

| | Positive | Negative | Total |
|---|---|---|---|
| Western blot | | | |
| Positive | 88 | 9 | 97 |
| Negative | 22 | 24 | 46 |
| Total | 110 | 33 | 143 |

Chi square analysis rejects the hypothesis of independence. The Western blot and immunofluorescence results are significantly related (p < 0.001).

Most Western blot positive sera, 88/110(80%), also were positive by IF (Table 2). Similarly 24/33sera (73%) that were negative by Western blot were also negative by immunofluorescence. Chi square analysis rejects the hypothesis of independence between antibody to the 68 kD protein and staining of supporting cells, indicating that the Western blot and immunofluorescence results are significantly related (p<0.001). Examples of staining have been published (Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995). This pattern is of particular interest because the patient sera replicate the staining pattern observed with the monoclonal antibody KHRI-3 (Zajic et al., “Monoclonal Antibodies To Inner Ear Antigens, I. Antigens Expressed By Supporting Cells Of The Guinea Pig Cochlea,” Hearing Res., 52:59-72, 1991; Ptok et al., “Monoclonal Antibodies To Inner Ear Antigens, II. Antigens Expressed In Sensory Cell Stereocilia,” Hearing Res., 57:79-90, 1991; Nair et al., “Monoclonal Antibody Induced Hearing Loss,” Hearing Res., 83:101-113, 1995; Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995).

### EXAMPLE VI

### Normal Control Serum Donors

Thus far, sera from 20 normal subjects ranging in age from 20 to 52 years of age have been tested by Western blot and immunofluorescence. None were positive for a 68 kD band, and only one stained supporting cells. All control subjects have normal hearing. Table 3 shows the frequency of antibodies to inner ear antigens in the sera of normal hearing individuals.

TABLE 3
Frequency Of Antibodies To Inner Ear Antigens In The Sera Of
Normal Hearing Individuals

| | Western Blot On Inner Ear Extract | | |
|---|---|---|---|
| Immunofluorescence | Number Of | Number Of | |
| To Supporting Cells | Sera Positive | Sera Negative | Totals |
| Number of sera positive | 0 | 1 | 1 |
| Number of sera negative | 0 | 19 | 19 |
| Totals | 0 | 20 | 20 |

### EXAMPLE VII

### Is The Inner Ear Specific Antigen HSP-70?

The nature of the target antigen of human autoimmune sera has been suggested to be heat shock protein, specifically HSP-70 (Rauch et al., “Serum Antibodies Against Heat Shock Proteins In Meniere's Disease,” Am. J. Otology, 16:648-652, 1995; Shin et al., “Comparison Of Anti-Heat Shock Protein 70 (Anti-hsp70) And Anti-68 kDa Inner Ear Protein In The Sera Of Patients With Meniere's Disease,” Laryngoscope, 107:222-227, 1997; Bloch et al., “Serum Antibodies To Heat Shock Protein 70 In Sensorineural Hearing Loss,” Arch. Otoloaryngol. Head Surg., 121:1167-1171, 1996). The null hypothesis is that the inner ear specific antigen and HSP-70 are one in the same. Reasons for proposing this hypothesis are the following: 1. HSP-70 is a ubiquitous protein and antibodies to it would not necessarily target the inner ear. 2. Antibodies to HSP-70 are found in the sera of many patients with other organ specific autoimmune diseases (Figuerdo et al., “Increased Serum Levels Of IgA Antibodies To hsp70 Protein In Patients With Diabetes Mellitus: Their Relationship With Vascular Complications,” Clin. Immunol. Immunopathol., 79:252-255, 1995; Shingai et al., “Autoantibody Against 70 kD Heat Shock Protein In Patients With Autoimmune Liver Diseases,” J. Hepatol., 23:382-390, 1995; Paggi et al., “Anti 70 kDa Heat Shock Protein Antibodies In Sera Of Patients Affected By Autoimmune And Non-Autoimmune Thyroid Diseases,” Endocr. Res., 21:555-567, 1995) yet it has been demonstrated that antibodies to more restricted antigens are the cause of specific autoimmune diseases (Anhalt et al., “Mechanisms Of Immunologic Injury. Pemphigus And Pemphigoid,” Arch Dermatol., 119:711-714, 1983; Wilkin, “Receptor Autoimmunity In Endocrine Disorders,” N. Engl. J. Med., 323:1318-1324, 1990; Amagai et al., “Autoantibodies Against The Amino-Terminal Cadherin-Like Binding Domain Of Pemphigus Vulgaris Antigen Are Pathogenic,” J. Clin. Invest., 90:919-926, 1992; Feldt-Rasmussen et al., “Anti-Thyroid Peroxidase Antibodies In Thyroid Disorders And Non-Thyroid Autoimmune Diseases,” Autoimmunity, 9:245-254, 1991; Disher et al., “Human Autoantibodies And Monoclonal Antibody KHRI-3 Bind To A Phylogenetically Conserved Inner Ear Supporting Cell Antigen,” Ann. NY, Acad, Sci., 630:253-65, 1997; Mappouras et al., “Antibodies To Acetylcholinesterase Cross-Reacting With Thyroglobulin In Myasthenia Gravis And Graves's Disease,” Clin. Exp. Immunol., 100:336-343, 1995; Chiovato et al., “Antibodies Producing Complement-Mediated Thyroid Cytotoxicity In Patients With Atrophic Or Goitrous Autoimmune Thyroiditis,” J. Clin. Endocrinol. Metab., 77:1700-1705, 1993; Li et al., “Catalytic Activity Of Anti-Thyroglobulin Antibodies,” J. Immunol., 154:3328-3332, 1995). 3. Our monoclonal antibody KHRI-3 can precipitate an antigen from inner ear extracts that reacts strongly with AISNHL patients' sera, but purified KHRI-3 does not precipitate a protein from these extracts that binds antibodies to HSP-70. 4. KHRI-3 and Western blot positive AISNHL human sera stain inner ear tissues with a similar pattern, but highly purified, high titered KHRI-3 antibody concentrated from hybridoma supernatant or produced in a bioreactor does not stain HSP-70 protein on Western blots.

Human sera are complex mixtures of thousands of antibodies. Sera may contain antibodies to both HSP-70 and to specific auto-antigen and those antigens can be completely distinct (Scheini et al., “Islet Cell And Glutamic Acid Decarboxylase Antibodies And Heat-Shock Protein 65 Responses In Children With Newly Diagnosed Insulin-Dependent Diabetes Mellitus,” Immunol. Lett., 49:123-126, 1996). This can happen even with highly specific antibody preparations, if there is a mixture of antibodies. For example, if we produce KHRJ-3 antibody in mice as ascites fluid this preparation will bind to HSP-70 protein because the mice produce anti-heat shock protein antibodies that contaminate the ascites fluid. AISNHL patients may have antibodies to HSP-70 and to an inner ear specific antigen, yet the inner ear specific antibody is likely to be the culprit in causing damage to the ear. Another alternative is that antibodies to an epitope on one protein that is broadly expressed (e.g., HSP-70) may also be expressed in another protein that is organ specific. The heat shock proteins (HSPs) are members of a family of molecular chaperons. These proteins serve to hold some newly synthesized proteins in the appropriate conformation until the nascent protein is mated to a protein or proteins with which it typically forms dimers or multimers. Similarly, when cells or organisms are placed under stress synthesis of HSPs increases presumably to protect critical proteins from denaturation. Thus, antibodies to HSPs may be produced as a result of release of cellular contents of damaged cells under the same stress (i.e., infection, trauma, etc.) that leads to formation of antibodies to inner ear specific antigens.

### EXAMPLE VIII

### The KHRI-3 Antigen And Heat Shock Protein Are Distinct

We have shown that monoclonal antibody to HSP-70 does not stain the guinea pig organ of Corti. These experiments were performed using immunofluorescence studies on surface preparations, both with and without tissue permeabilization. This difference in tissue expression makes it distinct from KHRI-3, which does bind to supporting cells on surface preparations in vivo and in vitro. HSP-70 is present in the inner ear since we can immunoprecipitate HSP-70 and detect it on Western blots using anti-HSP antibody. When proteins immunoprecipitated from guinea pig inner ear by anti HSP-70 and highly purified KHRI-3 Mab were Western blotted, KHRI-3 stained only the protein it precipitated and not HSP-70. Similarly, anti-HSP-70 stained only the 70 kD protein it precipitated and not the protein precipitated by KHRI-3 (Nair et al., Association for Research in Otolaryngology Feb. 15-19, 1998).

### EXAMPLE IX

### Human Inner Ear Absorbs Human Antibodies That Bind To Guinea Pig Inner Ear

To determine if the antigen detected by patient sera in guinea pig inner ear tissue is present in human inner ear, absorption analysis was performed. Inner ear tissue removed from patients undergoing ablative inner ear surgery was used as the source of inner ear antigen, and white and red blood cells from the same donors were used as histocompatibility and blood group antigen controls. Equal volumes of either packed blood cells or inner ear tissue were mixed with an aliquot of immunofluorescence (IF) positive patient sera and then retested on guinea pig inner ear substrate. Absorption with inner ear but not blood cells removed the antibody reactivity to guinea pig inner ear (Disher et al., “Human Autoantibodies And Monoclonal Antibody KHRI-3 Bind To A Phylogenetically Conserved Inner Ear Supporting Cell Antigen,” Ann. NY Acad. Sci., 630:336-343, 1995). In a second experiment not shown, a similar but less complete absorption was obtained.

### EXAMPLE X

### Western Blot Positive AISNHL Sera Stain An Inner Ear Protein Precipitated By KHRI-3

The similarity of IF and Western blot staining and our observations that KHRI-3 can cause hearing loss (Nair et al., “Monoclonal Antibody Induced Hearing Loss,” Hear. Res., 83:101-113, 1995; Nair et al., “In vivo Binding And Hearing Loss After Intracochlear Infusion Of KHRI-3 Antibody,” Hearing Res., 107:93-101, 1997) suggested that the human sera might bind to the same antigen as KHRI-3. To test this hypothesis, proteins precipitated from inner ear extracts by KHRI-3 were subjected to SDS PAGE, Western blotted and stained with patient sera. Thus far, 4/4patients' sera that were positive for the 68-70 kD band by Western blot also stained a 70 kD band in the material immunoprecipitated by KHRI-3. All three normal sera controls tested are negative. The KHRI-3 antibody binds best to non-reduced inner ear proteins. Under these conditions, we can usually resolve a doublet of 65 and 68 kD. KHRI-3 reacts poorly with reduced proteins but faint staining of the bands which shift to 68 and 70 kD under reducing conditions can be discerned. The human sera stain most strongly the upper band detected by KHRI-3 under reducing conditions. In contrast to KHRI-3, the human sera bind best to reduced proteins, indicating that the human antibodies and the murine antibody may identify different conformational epitopes on the same protein.

### EXAMPLE XI

### Antibody To Supporting Cells Is Significantly Associated With Hearing Improvement After Steroid Treatment

Although this is not a clinical study, we prospectively investigated response to treatment in a subset of patients for whom we had an acute phase serum specimen and objective measurement of hearing before and after steroid therapy. A total of 64 patients who were treated with steroids fit these criteria. Hearing improvement was based on the criteria of Moscicki et al. (Moscicki et al., “Correlation With Disease Activity And Response To Corticosteroid Treatment,” JAMA, 272-611-616, 1994) and was defined as greater than 10 dB threshold improvement at two consecutive frequencies and/or an improvement in speech discrimination of at least 20%. In our sample, 67% of the patients were antibody positive by Western blot and 75% were IF positive. Roughly, half of the patients (33/64) demonstrated clinical improvement and half (31/64) did not. Of those who were Western blot positive, 53% (23/43) improved; similarly, 48% (10/21) of the Western blot negative patients showed hearing improvement (p=0.66). In contrast, 60% (29/48) of patients who were IF positive had hearing improvement as compared to improvement in only 25% (4/16) of those who were IF negative (p=0.021). Table 4 shows the relationship between antibody to supporting cells and hearing improvement. In fact, 88% (29/33) of those who improved were positive for antibody to supporting cells by the IF assay and only 12.1% (4/33) of those who improved were IF negative. The presence of antibody to supporting cells is statistically significantly associated with hearing improvement after steroid therapy (Relative Risk: 2.4). Therefore, patients whose acute phase sera are IF positive are greater than two times more likely to experience improved hearing with steroid treatment than patients whose sera are IF negative. We suggest that this provides further support for the importance of the supporting cell antigen in the pathogenesis of autoimmune hearing loss. It is not surprising that a subset of the IF positive patients fail to improve since some may have already developed irreversible damage at the time therapy was initiated.

TABLE 4
Relationship Between Antibody To Supporting Cells And
Hearing Improvement

| | Hearing | No Hearing | |
|---|---|---|---|
| | Improvement | Improvement | Total |
| IF Positive | 29 (60%) | 19 (40%) | 48 (100%) |
| IF Negative | 4 (25%) | 12 (75%) | 16 (100%) |
| Total | 33 | 31 | 64 |

The difference is statistically significant, p = 0.021 (Fisher Exact Test).

1. A substantially purified glycoprotein from the inner-ear organ of Corti reactive with KHRI-3 monoclonal antibody.

2. A composition comprising guinea pig organ of Corti and sera from patients suspected of having AISNHL.

3. A composition comprising extracts from guinea pig organ of Corti, the KHRI-3 monoclonal antibody and sera from patients suspected of having AISNHL.

4. A method to detect antibodies in a patient's sera, comprising:
a. exposing guinea pig organ of Corti to patient's sera, said sera comprising antibodies; and
b. visualizing bound antibody.

5. A method to detect antibodies in a patient's sera comprising:
a. preparing an extract from guinea pig organ of Corti;
b. contacting the extract with KRHI-3 monoclonal antibody;
c. isolating an antigen-antibody complex; and
d. detecting the precipitated antigen with sera from patients suspected of having AISNHL.

6. The method of claim 5, further comprising detecting the antigen with sera from patients suspected of having AISNHL.
